(19) United States
(12) Patent Application Publication
(10) Pub. No.: US 2017/0044107 A1
(43) Pub. Date: Feb. 16, 2017

(54) Inhibitors of JMJD2C as Anticancer Agents

(71) Applicant: The J. David Gladstone Institutes, San Francisco, CA (US)

(72) Inventors: Min Xie, Daly City, CA (US); Sheng Ding, Orinda, CA (US)

(21) Appl. No.: 15/306,460

(22) PCT Filed: Apr. 21, 2015
(86) PCT No.: PCT/US15/26933

§ 371 Date: Oct. 24, 2016

Related U.S. Application Data
(60) Provisional application No. 61/985,221, filed on Apr. 28, 2014.

(51) Int. Cl. C07D 215/26; C07D 401/12

(52) U.S. Cl. CPC C07D 215/26; C07D 401/12

(57) ABSTRACT

The present disclosure provides compounds, pharmaceutical compositions and related methods for the treatment of cancer, e.g., castration-resistant prostate cancer (CRPC). Specifically, the present disclosure provides a series of 8-hydroxyquinoline derivatives which show cytotoxic effects on androgen-independent prostate cancer cells.

### CROSS REFERENCE

This application claims the benefit of U.S. Provisional Application No. 61/985,221 filed Apr. 28, 2014, the contents of which are incorporated by reference herein in their entirety.

### INTRODUCTION

Historically, patients diagnosed with castration-resistant prostate cancer (CRPC) have had poor survival rates. Despite significant advances in the treatment of CRPC in recent years, there remains a need in the art for additional therapeutic treatment options. The human histone demethylases of the KDM4 (JMJD2) family have been associated with diseases such as prostate and breast cancer. Accordingly, these enzymes are considered oncogenes, the selective inhibition of which may provide a possible therapeutic approach for the treatment of cancer. 8-hydroxyquinolines have been implicated as possible inhibitors of KDM4 activity. The present disclosure provides related methods and compositions for the treatment of disease, with particular applicability to the treatment of cancers, such as CRPC.

### SUMMARY

The present disclosure provides compounds, pharmaceutical compositions and related methods for the treatment of cancer, e.g., castration-resistant prostate cancer (CRPC). Specifically, the present disclosure provides a series of 8-hydroxyquinoline derivatives which show cytotoxic effects on androgen-independent prostate cancer cells in vitro. Pharmaceutical compositions containing these compounds are disclosed as are methods of treating cancers, such as CRPC, using these compounds.

Other aspects and embodiments will be readily apparent to the ordinarily skilled artisan upon reading the present specification.

### DEFINITIONS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications and applications mentioned herein are incorporated by reference to disclose and describe the methods and/or materials in connection with which the publications are cited. To the extent any of the applications or publications incorporated by reference herein conflict with the instant disclosure, the instant disclosure controls.

It must be noted that as used herein and in the appended claims, the singular forms “a”, “and”, and “the” include plural referents unless the context clearly dictates otherwise. Thus, for example, reference to “an 8-hydroxyquinoline derivative” includes a plurality of such derivatives and reference to the “pharmaceutical composition” includes reference to one or more pharmaceutical compositions and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR₁₀, —NR₁₀C(O)—, —C(O)NR₁₀— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″—where R′ is alkyl group as defined herein and R″ is alkylene, alkenylene or alkynylene group as defined herein.

The term “alkaryl” or “aralkyl” refers to the groups-alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)-—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, N R²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “aizido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)0-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic , provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂-moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “hydroxyamino” refers to the group —NHOH.

“Nitro” refers to the group —NO₂.

“Oxo” refers to the atom (═O).

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂OR⁷⁰, —OSO₂O⁻M⁺, —OSO₂R⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R^{80'}s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-pipcrazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂^{□}M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR ⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimcthylformamidc, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH-ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

The term “in combination with” as used herein refers to uses where, for example, a first therapy is administered during the entire course of administration of a second therapy; where the first therapy is administered for a period of time that is overlapping with the administration of the second therapy, e.g. where administration of the first therapy begins before the administration of the second therapy and the administration of the first therapy ends before the administration of the second therapy ends; where the administration of the second therapy begins before the administration of the first therapy and the administration of the second therapy ends before the administration of the first therapy ends; where the administration of the first therapy begins before administration of the second therapy begins and the administration of the second therapy ends before the administration of the first therapy ends; where the administration of the second therapy begins before administration of the first therapy begins and the administration of the first therapy ends before the administration of the second therapy ends. As such, “in combination” can also refer to regimen involving administration of two or more therapies. “In combination with” as used herein also refers to administration of two or more therapies which may be administered in the same or different formulations, by the same or different routes, and in the same or different dosage form type.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

“Subject”, “host” and “patient” are used interchangeably herein, to refer to an animal, human or non-human, amenable to therapy according to the methods of the disclosure or to which compound or composition according to the present disclosure may be administered to achieve a desired effect. Generally, the subject is a mammalian subject.

### DETAILED DESCRIPTION OF EXEMPLARY EMBODIMENTS

The present disclosure provides compounds, pharmaceutical compositions and related methods for the treatment of cancer; e.g., castration-resistant prostate cancer (CRPC); and/or the inhibition of histone demethylase JMJD2C. Specifically, the present disclosure provides a series of 8-hydroxyquinoline derivatives which show cytotoxic effects on androgen-independent prostate cancer cells. Particular compounds and pharmaceutical compositions of the present disclosure show activity as inhibitors of the histone demethylase JMJD2C. The human JMJD2C gene (NCBI Gene ID: 23081), also known as lysine (K)-specific demethylase 4C (KDM4C), is a member of the Jumonji domain 2 (JMJD2) family and encodes a protein with one JmjC domain, one JmjN domain, two PHD-type zinc fingers, and two Tudor domains. This nuclear protein functions as a trimethylation-specific demethylase, converting specific trimethylated histone residues to the dimethylated form. As discussed previously herein, human histone demethylases of the KDM4 (JMJD2) family have been associated with diseases such as prostate and breast cancer. Accordingly, these enzymes are considered oncogenes, the selective inhibition of which may provide a possible therapeutic approach for the treatment of cancer. The present disclosure provides related methods and compositions for the treatment of disease, with particular applicability to the treatment of cancers, such as CRPC.

The following substituents and values are intended to provide representative examples of various aspects and embodiments. These representative values are intended to further define and illustrate such aspects and embodiments and are not intended to exclude other embodiments or to limit the scope of the present disclosure. In this regard, the representation that a particular value or substituent is preferred is not intended in any way to exclude other values or substituents from the present disclosure unless specifically indicated.

These compounds may contain one or more chiral centers and therefore, the embodiments are directed to racemic mixtures; pure stereoisomers (i.e., enantiomers or diastereomers); stereoisomer-enriched mixtures and the like unless otherwise indicated. When a particular stereoisomer is shown or named herein, it will be understood by those skilled in the art that minor amounts of other stereoisomers may be present in the compositions unless otherwise indicated, provided that the desired utility of the composition as a whole is not eliminated by the presence of such other isomers.

Embodiments of the present disclosure include compounds of Formula I, shown below. Pharmaceutical compositions and methods of the present disclosure may also include compounds of Formula I.

### Formula I

Embodiments of the present disclosure include a compound of Formula I:

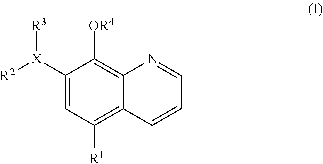

wherein

X is CH or a bond;

R¹ is selected from H, alkyl and substituted alkyl;

R² is selected from H, alkyl, substituted alkyl, amino, substituted amino, cycloalkyl, substituted cycloalkyl, heterocyclyl and substituted heterocyclyl;

R³is selected from H, alkyl, substituted alkyl, cycloalkyl, substituted cycloalkyl, heterocyclyl, substituted heterocyclyl, aryl, substituted aryl, heteroaryl and substituted heteroaryl; and

R⁴ is selected from H, alkyl and substituted alkyl,

or a salt or stereoisomer thereof.

In certain embodiments, if X is a bond, then R² is H, R³ is absent and R¹ is aminoalkyl or substituted aminoalkyl.

In certain embodiments, if R³ is heteroaryl or substituted aryl, then R⁴ is alkyl or substituted alkyl.

In certain embodiments, X is CH or a bond. In certain embodiments, X is CH. In certain embodiments, X is a bond. In certain embodiments, if X is a bond, then R² is H. In certain embodiments, if X is a bond, then R³ is absent. In certain embodiments, if X is a bond, then R² is H and R³ is absent. In certain embodiments, if X is a bond, then R¹ is alkyl or substituted alkyl. In certain embodiments, if X is a bond, then R¹ is alkyl, such as C₁₋₆ alkyl, or C₁₋₃ alkyl. In certain embodiments, if X is a bond, then R¹ is substituted alkyl, such as substituted C₁₋₆ alkyl, or substituted C₁₋₃ alkyl (e.g., substituted methyl, substituted ethyl or substituted propyl). In certain embodiments, if X is a bond, then R¹ is substituted methyl. In certain embodiments, if X is a bond, then R¹ is alkyl substituted with one or more amino or substituted amino groups (e.g., R¹ is aminoalkyl or substituted aminoalkyl). In certain embodiments, if X is a bond, then R¹ is aminoalkyl. In certain embodiments, if X is a bond, then R¹ is substituted aminoalkyl. For example, if X is a bond, then R¹ may be substituted aminoalkyl, where the amino is substituted with alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl or substituted heteroaryl. In some instances, if X is a bond, then R¹ may be substituted aminoalkyl, where the amino is substituted with alkyl or substituted alkyl. In some instances, if X is a bond, then R¹ may be substituted aminoalkyl, where the amino is substituted with substituted alkyl, such as a C₁₋₆ or C₁₋₃ alkyl substituted with cycloalkyl, substituted cycloalkyl, heterocyclyl, substituted heterocyclyl, aryl, substituted aryl, heteroaryl or substituted heteroaryl. In some instances, if X is a bond, then R¹ may be substituted aminoalkyl, where the amino is substituted with substituted alkyl, such as a C₁₋₃ alkyl (e.g., ethylene) substituted with heterocyclyl or substituted heterocyclyl (e.g., morpholinyl).

In certain embodiments, R¹ is selected from H, alkyl and substituted alkyl. In certain embodiments, R¹ is H. In certain embodiments, R¹ is alkyl or substituted alkyl. In certain embodiments, R¹ is alkyl, such as C₁₋₆ alkyl, or C₁₋₃ alkyl. In certain embodiments, R¹ is substituted alkyl, such as substituted C₁₋₆ alkyl, or substituted C₁₋₃ alkyl (e.g., substituted methyl, substituted ethyl or substituted propyl). In certain embodiments, R¹ is substituted methyl. In certain embodiments, R¹ is alkyl substituted with one or more amino or substituted amino groups (e.g., R¹ is aminoalkyl or substituted aminoalkyl). In certain embodiments, R¹ is aminoalkyl. In certain embodiments, R¹ is substituted aminoalkyl. For example, R¹ may be substituted aminoalkyl, where the amino is substituted with alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl or substituted heteroaryl. In some instances, R¹ is substituted aminoalkyl, where the amino is substituted with alkyl or substituted alkyl. In some instances, R¹ is substituted aminoalkyl, where the amino is substituted with substituted alkyl, such as a C₁₋₆ or C₁₋₃ alkyl substituted with cycloalkyl, substituted cycloalkyl, heterocyclyl, substituted heterocyclyl, aryl, substituted aryl, heteroaryl or substituted heteroaryl. In some instances, R¹ is substituted aminoalkyl, where the amino is substituted with substituted alkyl, such as a C₁₋₃ alkyl (e.g., ethylene) substituted with heterocyclyl or substituted heterocyclyl (e.g., morpholinyl).

In certain embodiments, R² is selected from H, alkyl, substituted alkyl, amino, substituted amino, cycloalkyl, substituted cycloalkyl, heterocyclyl and substituted heterocyclyl. In certain embodiments, R² is H. In certain embodiments, R² is alkyl or substituted alkyl. In certain embodiments, R² is alkyl, such as C₁₋₆ alkyl, or C₁₋₃ alkyl. In certain embodiments, R² is substituted alkyl, such as substituted C₁₋₆ alkyl, or substituted C₁₋₃ alkyl. In certain embodiments, R² is amino or substituted amino. In certain embodiments, R² is amino. In certain embodiments, R² is substituted amino (e.g., a secondary amino or a tertiary amino). In certain embodiments, R² is a substituted amino, where the amino is substituted with alkyl, substituted alkyl, cycloalkyl, substituted cycloalkyl, heterocyclyl, substituted heterocyclyl, aryl, substituted aryl, heteroaryl or substituted heteroaryl. In certain embodiments, R² is a substituted amino, where the amino is substituted with alkyl or substituted alkyl. In certain embodiments, R² is a substituted amino, where the amino is substituted with alkyl, such as C₁₋₆ alkyl, or C₁₋₃ alkyl. In certain embodiments, R² is a substituted amino, where the amino is substituted with alkyl, such as methyl, ethyl, propyl (e.g., n-propyl or isopropyl), butyl (e.g., n-butyl, sec-butyl, iso-butyl or tert-butyl), pentyl (e.g., n-pentyl, tert-pentyl, neo-pentyl, iso-pentyl, sec-pentyl or 3-pentyl), or hexyl (e.g., n-hexyl or branched-chain hexyl). In certain embodiments, R² is a substituted amino, where the amino is substituted with alkyl, such as iso-pentyl. In certain embodiments, R² is a substituted amino, where the amino is substituted with a substituted alkyl, such as substituted C₁₋₆ alkyl, or substituted C₁₋₃ alkyl. In certain embodiments, R² is a substituted amino, where the amino is substituted with a substituted alkyl, where the alkyl is substituted with cycloalkyl, substituted cycloalkyl, heterocyclyl, substituted heterocyclyl, aryl, substituted aryl, heteroaryl or substituted heteroaryl. In certain embodiments, R² is a substituted amino, where the amino is substituted with a substituted alkyl, where the alkyl is substituted with heterocyclyl or substituted heterocyclyl (e.g., morpholinyl).

In certain embodiments, R² is cycloalkyl or substituted cycloalkyl. In certain embodiments, R² is cycloalkyl, such as C₃₋₁₀ cycloalkyl, C₃₋₈ cycloalkyl or C₃₋₆ cycloalkyl. In certain embodiments, R² is substituted cycloalkyl, such as substituted C₃₋₁₀ cycloalkyl, substituted C₃₋₈ cycloalkyl or substituted C₃₋₆ cycloalkyl. In certain embodiments, R² is substituted C₃₋₆ cycloalkyl, such as substituted cyclopentyl or substituted cyclohexyl. In certain embodiments, R² is heterocyclyl or substituted heterocyclyl. In certain embodiments, R² is heterocyclyl, such as C₃₋₁₀ cycloalkyl, C₃₋₈ cycloalkyl or C₃₋₆ cycloalkyl, where one or more carbon atoms in the ring is selected from O, N or S. Examples of heterocyclyl groups include, but are not limited to pyrrolidinyl, pyrazolidinyl, imidazolidinyl, imidazolinyl, piperidinyl, piperazinyl, morpholinyl, tetrahydrofuranyl, oxazolidinyl, and the like. In certain embodiments, R² is piperazinyl. In certain embodiments, R² is substituted heterocyclyl such as substituted C₃₋₁₀ cycloalkyl, substituted C₃₋₈ cycloalkyl or substituted C₃₋₆ cycloalkyl, where one or more carbon atoms in the ring is selected from O, N or S. In certain embodiments, R² is substituted C₃₋₆ heterocyclyl, such as substituted cyclopentyl or substituted cyclohexyl, where one or more carbon atoms in the ring is selected from O, N or S. Examples of substituted heterocyclyls include, but are not limited to substituted pyrrolidinyl, substituted pyrazolidinyl, substituted imidazolidinyl, substituted imidazolinyl, substituted piperidinyl, substituted piperazinyl, substituted morpholinyl, substituted tetrahydrofuranyl, substituted oxazolidinyl, and the like. In certain embodiments, R² is substituted piperazinyl. In some instances, R² is a substituted heterocycyl, where the heterocyclyl is substituted with one or more alkyl, substituted alkyl, cycloalkyl, substituted cycloalkyl, heterocyclyl, substituted heterocyclyl, aryl, substituted aryl, heteroaryl or substituted heteroaryl. In some instances, R² is a substituted heterocycyl, where the heterocyclyl is substituted with aryl, substituted aryl, heteroaryl or substituted heteroaryl. In some instances, R² is a substituted heterocycyl, where the heterocyclyl is substituted with aryl or substituted aryl. In some instances, R² is a substituted heterocycyl, where the heterocyclyl is substituted with heteroaryl or substituted heteroaryl, such as, but not limited to, pyrrolyl, pyrazolyl, imidazolyl, pyridinyl, pyrazinyl, pyrimidinyl, furanyl, and the like. In some instances, R² is a substituted heterocycyl, where the heterocyclyl is substituted with pyridinyl. In some instances, R² is a substituted piperazinyl, where the piperazinyl is substituted with pyridinyl.

In certain embodiments, R³ is selected from H, alkyl, substituted alkyl, cycloalkyl, substituted cycloalkyl, heterocyclyl, substituted heterocyclyl, aryl, substituted aryl, heteroaryl and substituted heteroaryl. In certain embodiments, R³ is H. In certain embodiments, R³ is alkyl or substituted alkyl. In certain embodiments, R³ is alkyl, such as C₁₋₆ alkyl, or C₁₋₃ alkyl. In certain embodiments, R³ is substituted alkyl, such as substituted C₁₋₆ alkyl, or substituted C₁₋₃ alkyl. In certain embodiments, R³ is cycloalkyl or substituted cycloalkyl. In certain embodiments, R³ is cycloalkyl, such as C₃₋₁₀ cycloalkyl, C₃₋₈ cycloalkyl or C₃₋₆ cycloalkyl. In certain embodiments, R³ is substituted cycloalkyl, such as substituted C₃₋₁₀ cycloalkyl, substituted C₃₋₈ cycloalkyl or substituted C₃₋₆ cycloalkyl. In certain embodiments, R³ is heterocyclyl or substituted heterocyclyl. In certain embodiments, R³ is heterocyclyl such as C₃₋₁₀ cycloalkyl, C₃₋₈ cycloalkyl or C₃₋₆ cycloalkyl, where one or more carbon atoms in the ring is selected from O, N or S. In certain embodiments, R³ is substituted heterocyclyl, such as substituted C₃₋₁₀ cycloalkyl, substituted C₃₋₈ cycloalkyl or substituted C₃₋₆ cycloalkyl, where one or more carbon atoms in the ring is selected from O, N or S. In certain embodiments, R³ is aryl or substituted aryl. In certain embodiments, R³ is aryl. In certain embodiments, R³ is substituted aryl. In certain embodiments, R³ is heteroaryl or substituted heteroaryl. In certain embodiments, R³ is heteroaryl, such as, but not limited to, pyrrolyl, pyrazolyl, imidazolyl, pyridinyl, pyrazinyl, pyrimidinyl, furanyl, and the like. In certain embodiments, R³ is furanyl. In certain embodiments, R³ is substituted heteroaryl.

In certain embodiments, if R³ is heteroaryl or substituted aryl, then R⁴ is alkyl or substituted alkyl. In certain embodiments, if R³ is heteroaryl, then R⁴ is alkyl or substituted alkyl. In certain embodiments, if R³ is heteroaryl, then R⁴ is alkyl, such as C₁₋₆ alkyl, or C₁₋₃ alkyl (e.g., methyl). In certain embodiments, if R³ is heteroaryl, then R⁴ is methyl. In certain embodiments, if R³ is heteroaryl, then R⁴ is substituted alkyl, such as substituted C₁₋₆ alkyl, or substituted C₁₋₃ alkyl. In certain embodiments, if R³ is substituted aryl, then R⁴ is alkyl or substituted alkyl. In certain embodiments, if R³ is substituted aryl, then R⁴ is alkyl, such as C₁₋₆ alkyl, or C₁₋₃ alkyl (e.g., methyl). In certain embodiments, if R³ is substituted aryl, then R⁴ is substituted alkyl, such as substituted C₁₋₆ alkyl, or substituted C₁₋₃ alkyl.

In certain embodiments, R⁴ is selected from H, alkyl and substituted alkyl. In certain embodiments, R⁴ is H. In certain embodiments, R⁴ is alkyl or substituted alkyl. In certain embodiments, R⁴ is alkyl, such as C₁₋₆ alkyl, or C₁₋₃ alkyl (e.g., methyl). In certain embodiments, R⁴ is methyl. In certain embodiments, R⁴ is substituted alkyl, such as substituted C₁₋₆ alkyl, or substituted C₁₋₃ alkyl.

In certain embodiments, X is CH and R¹ is H.

In certain embodiments, X is a bond, R² is H, R³ is absent and R¹ is aminoalkyl or substituted aminoalkyl. In certain embodiments, X is a bond, R² is H, R³ is absent and R¹ is aminoalkyl, such as a C₁₋₆ alkyl, or C₁₋₃ alkyl substituted with an amino group. In certain embodiments, X is a bond, R² is H, R³ is absent and R¹ is aminoalkyl, such as a methylene substituted with an amino group. In certain embodiments, X is a bond, R² is H, R³ is absent and R¹ is substituted aminoalkyl, such as a C₁₋₆ alkyl, or C₁₋₃ alkyl substituted with a substituted amino group. In certain embodiments, X is a bond, R² is H, R³ is absent and R¹ is substituted aminoalkyl, such as a methylene substituted with a substituted amino group. In certain embodiments, X is a bond, R² is H, R³ is absent and R¹ is substituted aminoalkyl, where the amino is substituted with alkyl or substituted alkyl. In some instances, X is a bond, R² is H, R³ is absent and R¹ is substituted aminoalkyl, where the amino is substituted with substituted alkyl, such as a C₁₋₆ or C₁₋₃ alkyl substituted with cycloalkyl, substituted cycloalkyl, heterocyclyl, substituted heterocyclyl, aryl, substituted aryl, heteroaryl or substituted heteroaryl. In some instances, X is a bond, R² is H, R³ is absent and R¹ is substituted aminoalkyl, where the amino is substituted with substituted alkyl, such as a C₁₋₃ alkyl (e.g., ethylene) substituted with heterocyclyl or substituted heterocyclyl (e.g., morpholinyl).

In certain embodiments, R¹ and R³ are each H and R² is selected from amino, substituted amino, cycloalkyl, substituted cycloalkyl, heterocyclyl and substituted heterocyclyl. In certain embodiments, R¹ and R³ are each H and R² is amino or substituted amino. In certain embodiments, R¹ and R³ are each H and R² is amino. In certain embodiments, R¹ and R³ are each H and R² is substituted amino (e.g., a secondary amino or a tertiary amino). In certain embodiments, R¹ and R³ are each H and R² is a substituted amino, where the amino is substituted with alkyl, substituted alkyl, cycloalkyl, substituted cycloalkyl, heterocyclyl, substituted heterocyclyl, aryl, substituted aryl, heteroaryl or substituted heteroaryl. In certain embodiments, R¹ and R³ are each H and R² is a substituted amino, where the amino is substituted with alkyl or substituted alkyl. In certain embodiments, R¹ and R³ are each H and R² is a substituted amino, where the amino is substituted with alkyl, such as C₁₋₆ alkyl, or C₁₋₃ alkyl. In certain embodiments, R¹ and R³ are each H and R² is a substituted amino, where the amino is substituted with alkyl, such as methyl, ethyl, propyl (e.g., n-propyl or isopropyl), butyl (e.g., n-butyl, sec-butyl, iso-butyl or tert-butyl), pentyl (e.g., n-pentyl, tert-pentyl, neo-pentyl, iso-pentyl, sec-pentyl or 3-pentyl), or hexyl (e.g., n-hexyl or branched-chain hexyl). In certain embodiments, R¹ and R³ are each H and R² is a substituted amino, where the amino is substituted with alkyl, such as iso-pentyl. In certain embodiments, R¹ and R³ are each H and R² is a substituted amino, where the amino is substituted with a substituted alkyl, such as substituted C₁₋₆ alkyl, or substituted C₁₋₃ alkyl. In certain embodiments, R¹ and R³ are each H and R² is a substituted amino, where the amino is substituted with a substituted alkyl, where the alkyl is substituted with cycloalkyl, substituted cycloalkyl, heterocyclyl, substituted heterocyclyl, aryl, substituted aryl, heteroaryl or substituted heteroaryl. In certain embodiments, R¹ and R³ are each H and R² is a substituted amino, where the amino is substituted with a substituted alkyl, where the alkyl is substituted with heterocyclyl or substituted heterocyclyl (e.g., morpholinyl).

In certain embodiments, R¹ and R³ are each H and R² is cycloalkyl or substituted cycloalkyl. In certain embodiments, R¹ and R³ are each H and R² is cycloalkyl, such as C₃₋₁₀ cycloalkyl, C₃₋₈ cycloalkyl or C₃₋₆ cycloalkyl. In certain embodiments, R¹ and R³ are each H and R² is substituted cycloalkyl, such as substituted C₃₋₁₀ cycloalkyl, substituted C₃₋₈ cycloalkyl or substituted C₃₋₆ cycloalkyl. In certain embodiments, R¹ and R³ are each H and R² is substituted C₃₋₆ cycloalkyl, such as substituted cyclopentyl or substituted cyclohexyl. In certain embodiments, R¹ and R³ are each H and R² is heterocyclyl or substituted heterocyclyl. In certain embodiments, R¹ and R³ are each H and R² is heterocyclyl, such as C₃₋₁₀ cycloalkyl, C₃₋₈ cycloalkyl or C₃₋₆ cycloalkyl, where one or more carbon atoms in the ring is selected from O, N or S. Examples of heterocyclyls include, but are not limited to pyrrolidinyl, pyrazolidinyl, imidazolidinyl, imidazolinyl, piperidinyl, piperazinyl, morpholinyl, tetrahydrofuranyl, oxazolidinyl, and the like. In certain embodiments, R¹ and R³ are each H and R² is piperazinyl. In certain embodiments, R¹ and R³ are each H and R² is substituted heterocyclyl such as substituted C₃₋₁₀ cycloalkyl, substituted C₃₋₈ cycloalkyl or substituted C₃₋₆ cycloalkyl, where one or more carbon atoms in the ring is selected from O, N or S. In certain embodiments, R¹ and R³ are each H and R² is substituted C₃₋₆ heterocyclyl, such as substituted cyclopentyl or substituted cyclohexyl, where one or more carbon atoms in the ring is selected from O, N or S. Examples of substituted heterocyclyls include, but are not limited to substituted pyrrolidinyl, substituted pyrazolidinyl, substituted imidazolidinyl, substituted imidazolinyl, substituted piperidinyl, substituted piperazinyl, substituted morpholinyl, substituted tetrahydrofuranyl, substituted oxazolidinyl, and the like. In certain embodiments, R¹ and R³ are each H and R² is substituted piperazinyl. In some instances, R¹ and R³ are each H and R² is a substituted heterocycyl, where the heterocyclyl is substituted with one or more alkyl, substituted alkyl, cycloalkyl, substituted cycloalkyl, heterocyclyl, substituted heterocyclyl, aryl, substituted aryl, heteroaryl or substituted heteroaryl. In some instances, R¹ and R³ are each H and R² is a substituted heterocycyl, where the heterocyclyl is substituted with aryl, substituted aryl, heteroaryl or substituted heteroaryl. In some instances, R¹ and R³ are each H and R² is a substituted heterocycyl, where the heterocyclyl is substituted with aryl or substituted aryl. In some instances, R¹ and R³ are each H and R² is a substituted heterocycyl, where the heterocyclyl is substituted with heteroaryl or substituted heteroaryl, such as, but not limited to, pyrrolyl, pyrazolyl, imidazolyl, pyridinyl, pyrazinyl, pyrimidinyl, furanyl, and the like. In some instances, R¹ and R³ are each H and R² is a substituted heterocycyl, where the heterocyclyl is substituted with pyridinyl. In some instances, R¹ and R³ are each H and R² is a substituted piperazinyl, where the piperazinyl is substituted with pyridinyl.

In certain embodiments, R¹ is H, R² is amino or substituted amino, R³ is selected from aryl, substituted aryl, heteroaryl and substituted heteroaryl, and R⁴ is alkyl or substituted alkyl. In certain embodiments, R¹ is H, R² is amino, R³ is selected from aryl, substituted aryl, heteroaryl and substituted heteroaryl, and R⁴ is alkyl or substituted alkyl. In certain embodiments, R¹ is H, R² is substituted amino, R³ is selected from aryl, substituted aryl, heteroaryl and substituted heteroaryl, and R⁴ is alkyl or substituted alkyl. In certain embodiments, R¹ is H, R² is substituted amino, R³ is selected from aryl, substituted aryl, heteroaryl and substituted heteroaryl, and R⁴ is alkyl or substituted alkyl. In certain embodiments, R¹ is H, R² is substituted amino, R³ is selected from aryl, substituted aryl, heteroaryl and substituted heteroaryl, and R⁴ is alkyl. In certain embodiments, R¹ is H, R² is substituted amino, R³ is selected from aryl, substituted aryl, heteroaryl and substituted heteroaryl, and R⁴ is substituted alkyl. In certain embodiments, R¹ is H, R² is substituted amino, R³ is aryl or substituted aryl, and R⁴ is alkyl. In certain embodiments, R¹ is H, R² is substituted amino, R³ is heteroaryl or substituted heteroaryl, and R⁴ is alkyl. In certain embodiments, R¹ is H, R² is substituted amino, R³ is heteroaryl, and R⁴ is alkyl. In certain embodiments, R¹ is H, R² is substituted amino, R³ is substituted heteroaryl, and R⁴ is alkyl. In certain embodiments, where R³ is heteroaryl, R³ is selected from pyrrolyl, pyrazolyl, imidazolyl, pyridinyl, pyrazinyl, pyrimidinyl, and furanyl, and the like. In certain embodiments, R³ is furanyl. In certain embodiments, R¹ is H, R² is substituted amino, R³ is furanyl, and R⁴ is alkyl, such as C₁₋₆ alkyl, or C₁₋₃ alkyl (e.g., methyl). In certain embodiments, where R² is substituted amino, R² is an amino substituted with one or more alkyl, substituted alkyl, cycloalkyl, substituted cycloalkyl, heterocyclyl, substituted heterocyclyl, aryl, substituted aryl, heteroaryl or substituted heteroaryl. In certain embodiments, where R² is substituted amino, R² is an amino substituted with alkyl or substituted alkyl. In certain embodiments, where R² is substituted amino, R² is an amino substituted with alkyl, such as C₁₋₆ alkyl, or C₁₋₃ alkyl. In certain embodiments, where R² is substituted amino, R² is an amino substituted with a substituted alkyl, such as substituted C₁₋₆ alkyl, or substituted C₁₋₃ alkyl, where the substituted alkyl is substituted with one or more cycloalkyl, substituted cycloalkyl, heterocyclyl, substituted heterocyclyl, aryl, substituted aryl, heteroaryl, substituted heteroaryl, halo or oxo. In certain embodiments, where R² is substituted amino, R² is an amino substituted with a substituted alkyl, where the substituted alkyl is substituted with an oxo.

Particular compounds of interest of Formula I, and salts or solvates or stereoisomers thereof, include:

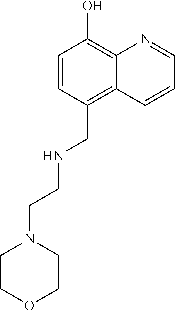 [SMILES: Oc1ccc(CNCCN2CCOCC2)c2cccnc12]

5-((2-morpholinoethylamino)methyl)quinolin-8-ol (Compound SD70-17);

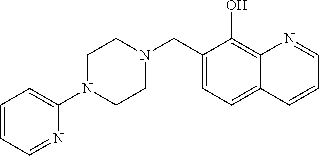 [SMILES: Oc1c(CN2CCN(c3ccccn3)CC2)ccc2cccnc12]

7-((4-(pyridin-2-yl)piperazin-1 -yl)methyl)quinolin-8-ol (Compound SD70-18);

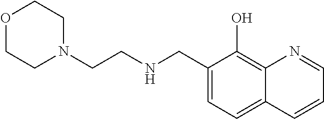 [SMILES: Oc1c(CNCCN2CCOCC2)ccc2cccnc12]

7-((2-morpholinoethylamino)methyl)quinolin-8-ol (Compound SD70-19); and

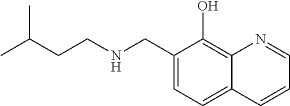 [SMILES: CC(C)CCNCc1ccc2cccnc2c1O]

7-((isopentylamino)methyl)quinolin-8-ol (Compound SD70-21).

Particular compounds of interest of Formula I, and salts or solvates or stereoisomers thereof, include:

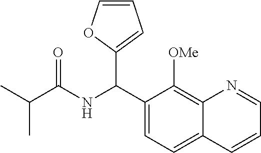 [SMILES: COc1c(C(NC(=O)C(C)C)c2ccco2)ccc2cccnc12]

N-(furan-2-yl(8-methoxyquinolin-7-yl)methyl)isobutyramide (Compound SD70-23).

In some instances, Compound SD70-23 finds use as a control compound, where the activities of other compounds of the present disclosure may be compared to the level of activity (or substantial lack thereof) of Compound SD70-23. As such, in certain embodiments, particular compounds and pharmaceutical compositions of the present disclosure, which show activity as inhibitors of histone demethylase JMJD2C and/or cytotoxic effects on androgen-independent prostate cancer cells, do not include Compound SD70-23.

Embodiments of the present disclosure include a compound of Formula Ia:

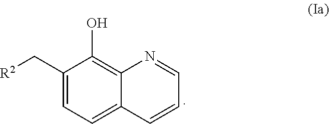

In certain embodiments, R² is selected from amino, substituted amino, heterocyclyl and substituted heterocyclyl. In certain embodiments, R² is amino. In certain embodiments, R² is substituted amino (e.g., a secondary amino or a tertiary amino). In certain embodiments, R² is a substituted amino, where the amino is substituted with alkyl, substituted alkyl, cycloalkyl, substituted cycloalkyl, heterocyclyl, substituted heterocyclyl, aryl, substituted aryl, heteroaryl or substituted heteroaryl. In certain embodiments, R² is a substituted amino, where the amino is substituted with alkyl or substituted alkyl. In certain embodiments, R² is a substituted amino, where the amino is substituted with alkyl, such as C₁₋₆ alkyl, or C₁₋₃ alkyl. In certain embodiments, R² is a substituted amino, where the amino is substituted with alkyl, such as methyl, ethyl, propyl (e.g., n-propyl or isopropyl), butyl (e.g., n-butyl, sec-butyl, iso-butyl or tert-butyl), pentyl (e.g., n-pentyl, tert-pentyl, neo-pentyl, iso-pentyl, sec-pentyl or 3-pentyl), or hexyl (e.g., n-hexyl or branched-chain hexyl). In certain embodiments, R² is a substituted amino, where the amino is substituted with alkyl, such as iso-pentyl. In certain embodiments, R² is a substituted amino, where the amino is substituted with a substituted alkyl, such as substituted C₁₋₆ alkyl, or substituted C₁₋₃ alkyl. In certain embodiments, R² is a substituted amino, where the amino is substituted with a substituted alkyl, where the alkyl is substituted with cycloalkyl, substituted cycloalkyl, heterocyclyl, substituted heterocyclyl, aryl, substituted aryl, heteroaryl or substituted heteroaryl. In certain embodiments, R² is a substituted amino, where the amino is substituted with a substituted alkyl, where the alkyl is substituted with heterocyclyl or substituted heterocyclyl (e.g., morpholinyl).

Particular compounds of interest of Formula Ia, and salts or solvates or stereoisomers thereof, include:

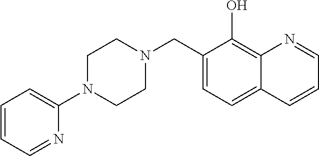 [SMILES: Oc1c(CN2CCN(c3ccccn3)CC2)ccc2cccnc12]

7-((4-(pyridin-2-yl)piperazin-1-yl)methyl)quinolin-8-ol (Compound SD70-18);

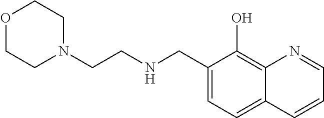 [SMILES: Oc1c(CNCCN2CCOCC2)ccc2cccnc12]

7-((2-morpholinoethylamino)methyl)quinolin-8-ol (Compound SD70-19); and

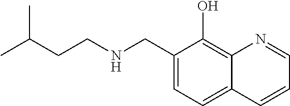 [SMILES: CC(C)CCNCc1ccc2cccnc2c1O]

7-((isopentylamino)methyl)quinolin-8-ol (Compound SD70-21).

Embodiments of the present disclosure include a compound of Formula Ib:

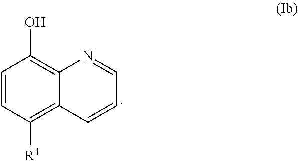

In certain embodiments, R¹ is aminoalkyl or substituted aminoalkyl (e.g., alkyl substituted with one or more amino or substituted amino groups). In certain embodiments, R¹ is aminoalkyl. In certain embodiments, R¹ is substituted aminoalkyl. For example, R¹ may be substituted aminoalkyl, where the amino is substituted with alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl or substituted heteroaryl. In some instances, R¹ is substituted aminoalkyl, where the amino is substituted with alkyl or substituted alkyl. In some instances, R¹ is substituted aminoalkyl, where the amino is substituted with substituted alkyl, such as a C₁₋₆ or C₁₋₃ alkyl substituted with cycloalkyl, substituted cycloalkyl, heterocyclyl, substituted heterocyclyl, aryl, substituted aryl, heteroaryl or substituted heteroaryl. In some instances, R¹ is substituted aminoalkyl, where the amino is substituted with substituted alkyl, such as a C₁₋₃ alkyl (e.g., ethylene) substituted with heterocyclyl or substituted heterocyclyl (e.g., morpholinyl).

Particular compounds of interest of Formula Ib, and salts or solvates or stereoisomers thereof, include:

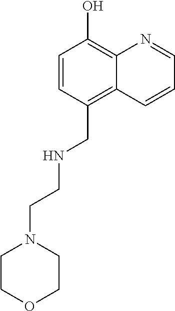 [SMILES: Oc1ccc(CNCCN2CCOCC2)c2cccnc12]

5-((2-morpholinoethylamino)methyl)quinolin-8-ol (Compound SD70-17).

Embodiments of the present disclosure include a compound of Formula Ic:

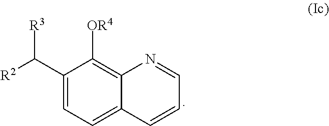

In certain embodiments, R² is amino or substituted amino, R³ is selected from aryl, substituted aryl, heteroaryl and substituted heteroaryl, and R⁴ is alkyl or substituted alkyl. In certain embodiments, R² is amino, R³ is selected from aryl, substituted aryl, heteroaryl and substituted heteroaryl, and R⁴ is alkyl or substituted alkyl. In certain embodiments, R² is substituted amino, R³ is selected from aryl, substituted aryl, heteroaryl and substituted heteroaryl, and R⁴ is alkyl or substituted alkyl. In certain embodiments, R² is substituted amino, R³ is selected from aryl, substituted aryl, heteroaryl and substituted heteroaryl, and R⁴ is alkyl or substituted alkyl. In certain embodiments, R² is substituted amino, R³ is selected from aryl, substituted aryl, heteroaryl and substituted heteroaryl, and R⁴ is alkyl. In certain embodiments, R² is substituted amino, R³ is selected from aryl, substituted aryl, heteroaryl and substituted heteroaryl, and R⁴ is substituted alkyl. In certain embodiments, R² is substituted amino, R³ is aryl or substituted aryl, and R⁴ is alkyl. In certain embodiments, R² is substituted amino, R³ is heteroaryl or substituted heteroaryl, and R⁴ is alkyl. In certain embodiments, R² is substituted amino, R³ is heteroaryl, and R⁴ is alkyl. In certain embodiments, R² is substituted amino, R³ is substituted heteroaryl, and R⁴ is alkyl. In certain embodiments, where R³ is heteroaryl, R³ is selected from pyrrolyl, pyrazolyl, imidazolyl, pyridinyl, pyrazinyl, pyrimidinyl, and furanyl, and the like. In certain embodiments, R³ is furanyl. In certain embodiments, R² is substituted amino, R³ is furanyl, and R⁴ is alkyl, such as C₁₋₆ alkyl, or C₁₋₃ alkyl (e.g., methyl). In certain embodiments, where R² is substituted amino, R² is an amino substituted with one or more alkyl, substituted alkyl, cycloalkyl, substituted cycloalkyl, heterocyclyl, substituted heterocyclyl, aryl, substituted aryl, heteroaryl or substituted heteroaryl. In certain embodiments, where R² is substituted amino, R² is an amino substituted with alkyl or substituted alkyl. In certain embodiments, where R² is substituted amino, R² is an amino substituted with alkyl, such as C₁₋₆ alkyl, or C₁₋₃ alkyl. In certain embodiments, where R² is substituted amino, R² is an amino substituted with a substituted alkyl, such as substituted C₁₋₆ alkyl, or substituted C₁₋₃ alkyl, where the substituted alkyl is substituted with one or more cycloalkyl, substituted cycloalkyl, heterocyclyl, substituted heterocyclyl, aryl, substituted aryl, heteroaryl, substituted heteroaryl, halo or oxo. In certain embodiments, where R² is substituted amino, R² is an amino substituted with a substituted alkyl, where the substituted alkyl is substituted with an oxo.

Particular compounds of interest of Formula Ic, and salts or solvates or stereoisomers thereof, include:

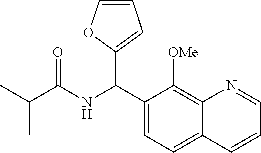 [SMILES: COc1c(C(NC(=O)C(C)C)c2ccco2)ccc2cccnc12]

N-(furan-2-yl(8-methoxyquinolin-7-yl)methyl)isobutyramide (Compound SD70-23).

In some instances, Compound SD70-23 finds use as a control compound, where the activities of other compounds of the present disclosure may be compared to the level of activity (or substantial lack thereof) of Compound SD70-23. As such, in certain embodiments, particular compounds and pharmaceutical compositions of the present disclosure, which show activity as inhibitors of histone demethylase JMJD2C and/or cytotoxic effects on androgen-independent prostate cancer cells, do not include Compound SD70-23.

Embodiments of the present disclosure include compounds of Formula II, shown below. Pharmaceutical compositions and methods of the present disclosure may also include compounds of Formula II.

### Formula II

Embodiments of the present disclosure include a compound of Formula II:

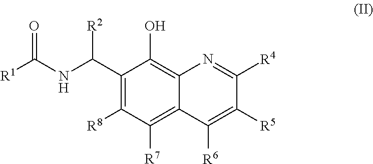

wherein

R⁷ is H or —(CH₂)ₙ—Y—R³, wherein n is an integer from 1 to 6, and Y is selected from O, NH, and NR⁹; and

R¹, R², R³, R⁴, R⁵, R⁶, R⁸ and R⁹ are each independently selected from H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl and substituted heteroaryl,or a salt or stereoisomer thereof.

In certain embodiments, R⁷ is H or —(CH₂)ₙ—Y—R³. In certain embodiments, R⁷ is H. In certain embodiments, R⁷ is —(CH₂)ₙ—Y—R³. In certain embodiments, n is an integer from 1 to 6 (e.g., 1, 2, 3, 4, 5 or 6). In certain embodiments, n is 1. In certain embodiments, n is 2. In certain embodiments, n is 3. In certain embodiments, n is 4. In certain embodiments, n is 5. In certain embodiments, n is 6. In certain embodiments, Y is selected from O, NH, and NR⁹. In certain embodiments, Y is O. In certain embodiments, Y is NH. In certain embodiments, Y is NR⁹.

In certain embodiments, R¹, R², R³, R⁴, R⁵, R⁶, R⁸ and R⁹ are each independently selected from H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl and substituted heteroaryl.

In certain embodiments, R¹ is selected from H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl and substituted heteroaryl. In certain embodiments, R¹ is H. In certain embodiments, R¹ is alkyl or substituted alkyl. In certain embodiments, R¹ is alkyl, such as C₁₋₆ alkyl or C₁₋₃ alkyl. In certain embodiments, R¹ is substituted alkyl, such as substituted C₁₋₆ alkyl or substituted C₁₋₃ alkyl. In certain embodiments, R¹ is alkenyl or substituted alkenyl. In certain embodiments, R¹ is alkynyl or substituted alkynyl. In certain embodiments, R¹ is cycloalkyl or substituted cycloalkyl. In certain embodiments, R¹ is cycloalkyl, such as C₃₋₁₀ cycloalkyl, C₃₋₈ cycloalkyl or C₃₋₆ cycloalkyl. In certain embodiments, R¹ is substituted cycloalkyl, such as substituted C₃₋₁₀ cycloalkyl, substituted C₃₋₈ cycloalkyl or substituted C₃₋₆ cycloalkyl. In certain embodiments, R¹ is heterocyclyl or substituted heterocyclyl. In certain embodiments, R¹ is heterocyclyl, such as C₃₋₁₀ heterocyclyl, C₃₋₈ heterocyclyl or C₃₋₆ heterocyclyl. In certain embodiments, R¹ is substituted heterocyclyl, such as substituted C₃₋₁₀ heterocyclyl, substituted C₃₋₈ heterocyclyl or substituted C₃₋₆ heterocyclyl. In certain embodiments, R¹ is aryl or substituted aryl. In certain embodiments, R¹ is aryl, such as C₃₋₁₀ aryl, C₄₋₈ aryl or C₅₋₆ aryl. In certain embodiments, R¹ is substituted aryl, such as substituted C₃₋₁₀ aryl, substituted C₄₋₈ aryl or substituted C₅₋₆ aryl. In certain embodiments, R¹ is heteroaryl or substituted heteroaryl. In certain embodiments, R¹ is heteroaryl, such as C₃₋₁₀ heteroaryl, C₄₋₈ heteroaryl or C₅₋₆ heteroaryl. In certain embodiments, R¹ is substituted heteroaryl, such as substituted C₃₋₁₀ heteroaryl, substituted C₄₋₈ heteroaryl or substituted C₅₋₆ heteroaryl.

In certain embodiments, R² is selected from H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl and substituted heteroaryl. In certain embodiments, R² is H. In certain embodiments, R² is alkyl or substituted alkyl. In certain embodiments, R² is alkyl, such as C₁₋₆ alkyl or C₁₋₃ alkyl. In certain embodiments, R² is substituted alkyl, such as substituted C₁₋₆ alkyl or substituted C₁₋₃ alkyl. In certain embodiments, R² is alkenyl or substituted alkenyl. In certain embodiments, R² is alkynyl or substituted alkynyl. In certain embodiments, R² is cycloalkyl or substituted cycloalkyl. In certain embodiments, R² is cycloalkyl, such as C₃₋₁₀ cycloalkyl, C₃₋₈ cycloalkyl or C₃₋₆ cycloalkyl. In certain embodiments, R² is substituted cycloalkyl, such as substituted C₃₋₁₀ cycloalkyl, substituted C₃₋₈ cycloalkyl or substituted C₃₋₆ cycloalkyl. In certain embodiments, R² is heterocyclyl or substituted heterocyclyl. In certain embodiments, R² is heterocyclyl, such as C₃₋₁₀ heterocyclyl, C₃₋₈ heterocyclyl or C₃₋₆ heterocyclyl. In certain embodiments, R² is substituted heterocyclyl, such as substituted C₃₋₁₀ heterocyclyl, substituted C₃₋₈ heterocyclyl or substituted C₃₋₆ heterocyclyl. In certain embodiments, R² is aryl or substituted aryl. In certain embodiments, R² is aryl, such as C₃₋₁₀ aryl, C₄₋₈ aryl or C₅₋₆ aryl. In certain embodiments, R² is substituted aryl, such as substituted C₃₋₁₀ aryl, substituted C₄₋₈ aryl or substituted C₅₋₆ aryl. In certain embodiments, R² is heteroaryl or substituted heteroaryl. In certain embodiments, R² is heteroaryl, such as C₃₋₁₀ heteroaryl, C₄₋₈ heteroaryl or C₅₋₆ heteroaryl. In certain embodiments, R² is substituted heteroaryl, such as substituted C₃₋₁₀ heteroaryl, substituted C₄₋₈ heteroaryl or substituted C₅₋₆ heteroaryl.

In certain embodiments, R³ is selected from H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl and substituted heteroaryl. In certain embodiments, R³ is H. In certain embodiments, R³ is alkyl or substituted alkyl. In certain embodiments, R³ is alkyl, such as C₁₋₆ alkyl or C₁₋₃ alkyl. In certain embodiments, R³ is substituted alkyl, such as substituted C₁₋₆ alkyl or substituted C₁₋₃ alkyl. In certain embodiments, R³ is alkenyl or substituted alkenyl. In certain embodiments, R³ is alkynyl or substituted alkynyl. In certain embodiments, R³ is cycloalkyl or substituted cycloalkyl. In certain embodiments, R³ is cycloalkyl, such as C₃₋₁₀ cycloalkyl, C₃₋₈ cycloalkyl or C₃₋₆ cycloalkyl. In certain embodiments, R³ is substituted cycloalkyl, such as substituted C₃₋₁₀ cycloalkyl, substituted C₃₋₈ cycloalkyl or substituted C₃₋₆ cycloalkyl. In certain embodiments, R³ is heterocyclyl or substituted heterocyclyl. In certain embodiments, R³ is heterocyclyl, such as C₃₋₁₀ heterocyclyl, C₃₋₈ heterocyclyl or C₃₋₆ heterocyclyl. In certain embodiments, R³ is substituted heterocyclyl, such as substituted C₃₋₁₀ heterocyclyl, substituted C₃₋₈ heterocyclyl or substituted C₃₋₆ heterocyclyl. In certain embodiments, R³ is aryl or substituted aryl. In certain embodiments, R³ is aryl, such as C₃₋₁₀ aryl, C₄₋₈ aryl or C₅₋₆ aryl. In certain embodiments, R³ is substituted aryl, such as substituted C₃₋₁₀ aryl, substituted C₄₋₈ aryl or substituted C₅₋₆ aryl. In certain embodiments, R³ is heteroaryl or substituted heteroaryl. In certain embodiments, R³ is heteroaryl, such as C₃₋₁₀ heteroaryl, C₄₋₈ heteroaryl or C₅₋₆ heteroaryl. In certain embodiments, R³ is substituted heteroaryl, such as substituted C₃₋₁₀ heteroaryl, substituted C₄₋₈ heteroaryl or substituted C₅₋₆ heteroaryl.

In certain embodiments, R⁴ is selected from H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl and substituted heteroaryl. In certain embodiments, R⁴ is H. In certain embodiments, R⁴ is alkyl or substituted alkyl. In certain embodiments, R⁴ is alkyl, such as C₁₋₆ alkyl or C₁₋₃ alkyl. In certain embodiments, R⁴ is substituted alkyl, such as substituted C₁₋₆ alkyl or substituted C₁₋₃ alkyl. In certain embodiments, R⁴ is alkenyl or substituted alkenyl. In certain embodiments, R⁴ is alkynyl or substituted alkynyl. In certain embodiments, R⁴ is cycloalkyl or substituted cycloalkyl. In certain embodiments, R⁴ is cycloalkyl, such as C₃₋₁₀ cycloalkyl, C3-8 cycloalkyl or C₃₋₆ cycloalkyl. In certain embodiments, R⁴ is substituted cycloalkyl, such as substituted C₃₋₁₀ cycloalkyl, substituted C₃₋₈ cycloalkyl or substituted C₃₋₆ cycloalkyl. In certain embodiments, R⁴ is heterocyclyl or substituted heterocyclyl. In certain embodiments, R⁴ is heterocyclyl, such as C₃₋₁₀ heterocyclyl, C₃₋₈ heterocyclyl or C₃₋₆ heterocyclyl. In certain embodiments, R⁴ is substituted heterocyclyl, such as substituted C₃₋₁₀ heterocyclyl, substituted C₃₋₈ heterocyclyl or substituted C₃₋₆ heterocyclyl. In certain embodiments, R⁴ is aryl or substituted aryl. In certain embodiments, R⁴ is aryl, such as C₃₋₁₀ aryl, C₄₋₈ aryl or C₅₋₆ aryl. In certain embodiments, R⁴ is substituted aryl, such as substituted C₃₋₁₀ aryl, substituted C₄₋₈ aryl or substituted C₅₋₆ aryl. In certain embodiments, R⁴ is heteroaryl or substituted heteroaryl. In certain embodiments, R⁴ is heteroaryl, such as C₃₋₁₀ heteroaryl, C₄₋₈ heteroaryl or C₅₋₆ heteroaryl. In certain embodiments, R⁴ is substituted heteroaryl, such as substituted C₃₋₁₀ heteroaryl, substituted C₄₋₈ heteroaryl or substituted C₅₋₆ heteroaryl.

In certain embodiments, R⁵ is selected from H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl and substituted heteroaryl. In certain embodiments, R⁵ is H. In certain embodiments, R⁵ is alkyl or substituted alkyl. In certain embodiments, R⁵ is alkyl, such as C₁₋₆ alkyl or C₁₋₃ alkyl. In certain embodiments, R⁵ is substituted alkyl, such as substituted C₁₋₆ alkyl or substituted C₁₋₃ alkyl. In certain embodiments, R⁵ is alkenyl or substituted alkenyl. In certain embodiments, R⁵ is alkynyl or substituted alkynyl. In certain embodiments, R⁵ is cycloalkyl or substituted cycloalkyl. In certain embodiments, R⁵ is cycloalkyl, such as C₃₋₁₀ cycloalkyl, C₃₋₈ cycloalkyl or C₃₋₆ cycloalkyl. In certain embodiments, R⁵ is substituted cycloalkyl, such as substituted C₃₋₁₀ cycloalkyl, substituted C₃₋₈ cycloalkyl or substituted C₃₋₆ cycloalkyl. In certain embodiments, R⁵ is heterocyclyl or substituted heterocyclyl. In certain embodiments, R⁵ is heterocyclyl, such as C₃₋₁₀ heterocyclyl, C₃₋₈ heterocyclyl or C₃₋₆ heterocyclyl. In certain embodiments, R⁵ is substituted heterocyclyl, such as substituted C₃₋₁₀ heterocyclyl, substituted C₃₋₈ heterocyclyl or substituted C₃₋₆ heterocyclyl. In certain embodiments, R⁵ is aryl or substituted aryl. In certain embodiments, R⁵ is aryl, such as C₃₋₁₀ aryl, C₄₋₈ aryl or C₅₋₆ aryl. In certain embodiments, R⁵ is substituted aryl, such as substituted C₃₋₁₀ aryl, substituted C₄₋₈ aryl or substituted C₅₋₆ aryl. In certain embodiments, R⁵ is heteroaryl or substituted heteroaryl. In certain embodiments, R⁵ is heteroaryl, such as C₃₋₁₀ heteroaryl, C₄₋₈ heteroaryl or C₅₋₆ heteroaryl. In certain embodiments, R⁵ is substituted heteroaryl, such as substituted C₃₋₁₀ heteroaryl, substituted C₄₋₈ heteroaryl or substituted C₅₋₆ heteroaryl.

In certain embodiments, R⁶ is selected from H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl and substituted heteroaryl. In certain embodiments, R⁶ is H. In certain embodiments, R⁶ is alkyl or substituted alkyl. In certain embodiments, R⁶ is alkyl, such as C₁₋₆ alkyl or C₁₋₃ alkyl. In certain embodiments, R⁶ is substituted alkyl, such as substituted C₁₋₆ alkyl or substituted C₁₋₃ alkyl. In certain embodiments, R⁶ is alkenyl or substituted alkenyl. In certain embodiments, R⁶ is alkynyl or substituted alkynyl. In certain embodiments, R⁶ is cycloalkyl or substituted cycloalkyl. In certain embodiments, R⁶ is cycloalkyl, such as C₃₋₁₀ cycloalkyl, C₃₋₈ cycloalkyl or C₃₋₆ cycloalkyl. In certain embodiments, R⁶ is substituted cycloalkyl, such as substituted C₃₋₁₀ cycloalkyl, substituted C₃₋₈ cycloalkyl or substituted C₃₋₆ cycloalkyl. In certain embodiments, R⁶ is heterocyclyl or substituted heterocyclyl. In certain embodiments, R⁶ is heterocyclyl, such as C₃₋₁₀ heterocyclyl, C₃₋₈ heterocyclyl or C₃₋₆ heterocyclyl. In certain embodiments, R⁶ is substituted heterocyclyl, such as substituted C₃₋₁₀ heterocyclyl, substituted C₃₋₈ heterocyclyl or substituted C₃₋₆ heterocyclyl. In certain embodiments, R⁶ is aryl or substituted aryl. In certain embodiments, R⁶ is aryl, such as C₃₋₁₀ aryl, C₄₋₈ aryl or C₅₋₆ aryl. In certain embodiments, R⁶ is substituted aryl, such as substituted C₃₋₁₀ aryl, substituted C₄₋₈ aryl or substituted C₅₋₆ aryl. In certain embodiments, R⁶ is heteroaryl or substituted heteroaryl. In certain embodiments, R⁶ is heteroaryl, such as C₃₋₁₀ heteroaryl, C₄₋₈ heteroaryl or C₅₋₆ heteroaryl. In certain embodiments, R⁶ is substituted heteroaryl, such as substituted C₃₋₁₀ heteroaryl, substituted C₄₋₈ heteroaryl or substituted C₅₋₆ heteroaryl.

In certain embodiments, R⁸ is selected from H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl and substituted heteroaryl. In certain embodiments, R⁸ is H. In certain embodiments, R⁸ is alkyl or substituted alkyl. In certain embodiments, R⁸ is alkyl, such as C₁₋₆ alkyl or C₁₋₃ alkyl. In certain embodiments, R⁸ is substituted alkyl, such as substituted C₁₋₆ alkyl or substituted C₁₋₃ alkyl. In certain embodiments, R⁸ is alkenyl or substituted alkenyl. In certain embodiments, R⁸ is alkynyl or substituted alkynyl. In certain embodiments, R⁸ is cycloalkyl or substituted cycloalkyl. In certain embodiments, R⁸ is cycloalkyl, such as C₃₋₁₀ cycloalkyl, C₃₋₈ cycloalkyl or C₃₋₆ cycloalkyl. In certain embodiments, R⁸ is substituted cycloalkyl, such as substituted C₃₋₁₀ cycloalkyl, substituted C₃₋₈ cycloalkyl or substituted C₃₋₆ cycloalkyl. In certain embodiments, R⁸ is heterocyclyl or substituted heterocyclyl. In certain embodiments, R⁸ is heterocyclyl, such as C₃₋₁₀ heterocyclyl, C₃₋₈ heterocyclyl or C₃₋₆ heterocyclyl. In certain embodiments, R⁸ is substituted heterocyclyl, such as substituted C₃₋₁₀ heterocyclyl, substituted C₃₋₈ heterocyclyl or substituted C₃₋₆ heterocyclyl. In certain embodiments, R⁸ is aryl or substituted aryl. In certain embodiments, R⁸ is aryl, such as C₃₋₁₀ aryl, C₄₋₈ aryl or C₅₋₆ aryl. In certain embodiments, R⁸ is substituted aryl, such as substituted C₃₋₁₀ aryl, substituted C₄₋₈ aryl or substituted C₅₋₆ aryl. In certain embodiments, R⁸ is heteroaryl or substituted heteroaryl. In certain embodiments, R⁸ is heteroaryl, such as C₃₋₁₀ heteroaryl, C₄₋₈ heteroaryl or C₅₋₆ heteroaryl. In certain embodiments, R⁸ is substituted heteroaryl, such as substituted C₃₋₁₀ heteroaryl, substituted C₄₋₈ heteroaryl or substituted C₅₋₆ heteroaryl. In certain embodiments, R⁹ is selected from H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl and substituted heteroaryl. In certain embodiments, R⁹ is H. In certain embodiments, R⁹ is alkyl or substituted alkyl. In certain embodiments, R⁹ is alkyl, such as C₁₋₆ alkyl or C₁₋₃ alkyl. In certain embodiments, R⁹ is substituted alkyl, such as substituted C₁₋₆ alkyl or substituted C₁₋₃ alkyl. In certain embodiments, R⁹ is alkenyl or substituted alkenyl. In certain embodiments, R⁹ is alkynyl or substituted alkynyl. In certain embodiments, R⁹ is cycloalkyl or substituted cycloalkyl. In certain embodiments, R⁹ is cycloalkyl, such as C₃₋₁₀ cycloalkyl, C₃₋₈ cycloalkyl or C₃₋₆ cycloalkyl. In certain embodiments, R⁹ is substituted cycloalkyl, such as substituted C₃₋₁₀ cycloalkyl, substituted C₃₋₈ cycloalkyl or substituted C₃₋₆ cycloalkyl. In certain embodiments, R⁹ is heterocyclyl or substituted heterocyclyl. In certain embodiments, R⁹ is heterocyclyl, such as C₃₋₁₀ heterocyclyl, C₃₋₈ heterocyclyl or C₃₋₆ heterocyclyl. In certain embodiments, R⁹ is substituted heterocyclyl, such as substituted C₃₋₁₀ heterocyclyl, substituted C₃₋₈ heterocyclyl or substituted C₃₋₆ heterocyclyl. In certain embodiments, R⁹ is aryl or substituted aryl. In certain embodiments, R⁹ is aryl, such as C₃₋₁₀ aryl, C₄₋₈ aryl or C₅₋₆ aryl. In certain embodiments, R⁹ is substituted aryl, such as substituted C₃₋₁₀ aryl, substituted C₄₋₈ aryl or substituted C₅₋₆ aryl. In certain embodiments, R⁹ is heteroaryl or substituted heteroaryl. In certain embodiments, R⁹ is heteroaryl, such as C₃₋₁₀ heteroaryl, C₄₋₈ heteroaryl or C₅₋₆ heteroaryl. In certain embodiments, R⁹ is substituted heteroaryl, such as substituted C₃₋₁₀ heteroaryl, substituted C₄₋₈ heteroaryl or substituted C₅₋₆ heteroaryl.

### General Synthetic Procedures

Many general references providing commonly known chemical synthetic schemes and conditions useful for synthesizing the disclosed compounds are available (see, e.g., Smith and March, March s Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, edE. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in The Peptides; Volume 3 (editors: E. Gross and J. Meienhofer), Academic Press, London and New York 1981, in “Methoden der organischen Chemie”, Houben-Weyl, 4^{th} edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. Exemplary synthetic methods for the 8-hydroxyquinoline derivatives described herein are described below. Those of skill in the art will also be able to readily adapt these methods for the synthesis of specific 8-hydroxyquinoline derivatives as described herein.

### General Synthetic Scheme for 8-Hydroxyquinoline Derivatives

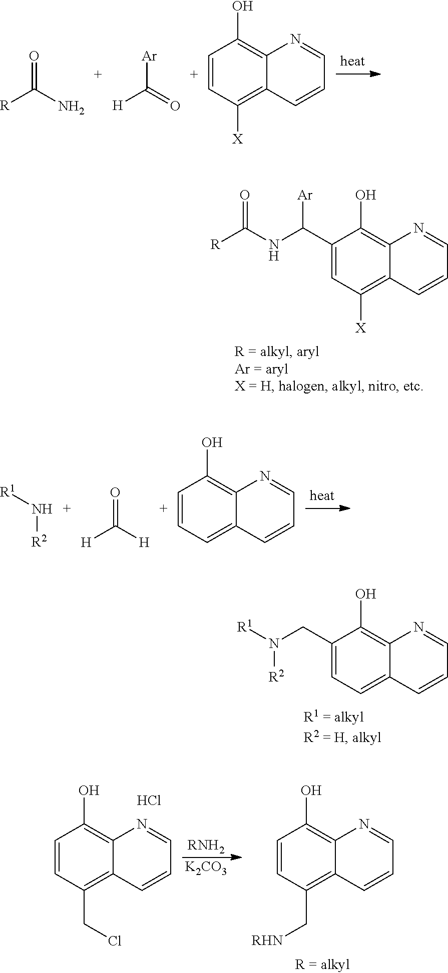

### Representative Synthesis of Compound SD70 and Derivatives Thereof

In certain embodiments, 8-hydroxyquinoline (1, 580 mg, 4.0 mmol), furfural (2, 768 mg, 8.0 mmol), and isobutyramide (3, 523 mg, 6.0 mmol) are mixed in ethanol (2 mL) in a glass, pressure tube. The mixtures are heated at 150° C. for 3 hours, cooled to room temperature, and concentrated under reduced pressure. The residue may be chromatographed on silica gel (hexane/ethyl acetate 6/1, 1/1) to afford SD70 as a solid. In certain embodiments, SD70 can be further purified by recrystallization in the mixtures of hexane/ethyl acetate (1/1).

### Pharmaceutical Compositions

In certain embodiments, the disclosed compounds are useful for the inhibition of histone demethylase JMJD2C activity and/or the treatment of a disease or disorder that is mediated through the activity of histone demethylase JMJD2C, such as prostate cancer. In certain embodiments, the disclosed compounds are useful for the treatment of cancer, e.g., the treatment of a castration-resistant prostate cancer (CRPC). Accordingly, pharmaceutical compositions comprising at least one disclosed compound are also described herein.

A pharmaceutical composition that includes a subject compound may be administered to a patient alone, or in combination with other supplementary active agents. The pharmaceutical compositions may be manufactured using any of a variety of processes, including, but not limited to, conventional mixing, dissolving, granulating, dragee-making, levigating, emulsifying, encapsulating, entrapping, lyophilizing, and the like. The pharmaceutical composition can take any of a variety of forms including, but not limited to, a sterile solution, suspension, emulsion, lyophilisate, tablet, pill, pellet, capsule, powder, syrup, elixir or any other dosage form suitable for administration.

A subject compound may be administered to a subject using any convenient means capable of resulting in the desired reduction in disease condition or symptom. Thus, a subject compound can be incorporated into a variety of formulations for therapeutic administration. More particularly, a subject compound can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, aerosols, and the like

Formulations for pharmaceutical compositions are described in, for example, Remingtons Pharmaceutical Sciences, by E. W. Martin, Mack Publishing Co., Easton, Pa., 19th Edition, 1995, which describes examples of formulations (and components thereof) suitable for pharmaceutical delivery of disclosed compounds. Pharmaceutical compositions that include at least one of the subject compounds can be formulated for use in human or veterinary medicine. Particular formulations of a disclosed pharmaceutical composition may depend, for example, on the mode of administration and/or on the location of the subject to be treated. In some embodiments, formulations include a pharmaceutically acceptable carrier in addition to at least one active ingredient, such as a subject compound. In other embodiments, other medicinal or pharmaceutical agents, for example, with similar, related or complementary effects on the disease or condition being treated can also be included as active ingredients in a pharmaceutical composition.

Pharmaceutically acceptable carriers useful for the disclosed methods and compositions may depend on the particular mode of administration being employed. For example, parenteral formulations may include injectable fluids, such as, but not limited to, pharmaceutically and physiologically acceptable fluids such as water, physiological saline, balanced salt solutions, aqueous dextrose, glycerol or the like as a vehicle. For solid compositions (e.g., powder, pill, tablet, or capsule forms), non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances (e.g., excipients), such as wetting or emulsifying agents, preservatives, and pH buffering agents and the like; for example, sodium acetate or sorbitan monolaurate. Other examples of excipients include, nonionic solubilizers, such as cremophor, or proteins, such as human serum albumin or plasma preparations.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) water (e.g., pyrogen-free water); (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The disclosed pharmaceutical compositions may be formulated as a pharmaceutically acceptable salt of a disclosed compound. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydroiodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, methyl sulfonic acid, salicylic acid, formic acid, trichloroacetic acid, trifluoroacetic acid, gluconic acid, asparagic acid, aspartic acid, benzenesulfonic acid, para-tolucncsulfonic acid, naphthalenesulfonic acid, and the like. In certain embodiments, the pharmaceutically acceptable salt includes formic acid. In certain embodiments, the pharmaceutically acceptable salt includes trifluoroacetic acid. Other suitable pharmaceutically acceptable salts are found in Remingtons Pharmaceutical Sciences, 17th Edition, Mack Publishing Company, Easton, Pa., 1985. A pharmaceutically acceptable salt may also serve to adjust the osmotic pressure of the composition.

A subject compound can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Such preparations can be used for oral administration.

A subject compound can be formulated into preparations for injection by dissolving, suspending or emulsifying the compound in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The preparation may also be emulsified or the active ingredient encapsulated in liposome vehicles. Formulations suitable for injection can be administered by an intravitreal, intraocular, intramuscular, subcutaneous, sublingual, or other route of administration, e.g., injection into the gum tissue or other oral tissue. Such formulations are also suitable for topical administration.

In some embodiments, a subject compound can be delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

A subject compound can be utilized in aerosol formulation to be administered via inhalation. A subject compound can be formulated into pressurized acceptable propellants such as dichlorodifluoromethane, propane, nitrogen and the like.

Furthermore, a subject compound can be made into suppositories by mixing with a variety of bases such as emulsifying bases or water-soluble bases. A subject compound can be administered rectally via a suppository. The suppository can include vehicles such as cocoa butter, carbowaxes and polyethylene glycols, which melt at body temperature, yet are substantially solid at room temperature.

The term “unit dosage form,” as used herein, refers to physically discrete units suitable as unitary dosages for human and animal subjects, each unit containing a predetermined quantity of a subject compound calculated in an amount sufficient to produce the desired effect in association with a pharmaceutically acceptable diluent, carrier or vehicle. The specifications for a subject compound depend on the particular compound employed and the effect to be achieved, and the pharmacodynamics associated with each compound in the host.

The dosage form of a disclosed pharmaceutical composition may be determined by the mode of administration chosen. For example, in addition to injectable fluids, topical or oral dosage forms may be employed. Topical preparations may include eye drops, ointments, sprays and the like. Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical compositions that include a subject compound may be formulated in unit dosage form suitable for individual administration of precise dosages. The amount of active ingredient administered may depend on the subject being treated, the severity of the affliction, and the manner of administration, and is known to those skilled in the art. In certain instances, the formulation to be administered contains a quantity of the compounds disclosed herein in an amount effective to achieve the desired effect in the subject being treated.

Each therapeutic compound can independently be in any dosage form, such as those described herein, and can also be administered in various ways, as described herein. For example, the compounds may be formulated together, in a single dosage unit (that is, combined together in one form such as capsule, tablet, powder, or liquid, etc.) as a combination product. Alternatively, when not formulated together in a single dosage unit, an individual subject compound may be administered at the same time as another therapeutic compound or sequentially, in any order thereof.

### Methods of Administration

Compounds disclose herein can inhibit histone demethylase JMJD2C activity. Accordingly, such compounds are useful for treating a disease or disorder that is mediated through the activity of histone demethylase JMJD2C in a subject. In certain embodiments, the subject compounds are useful for treating a disease or disorder that is associated with the histone demethylase JMJD2C activity in a subject, such as prostate cancer. Compounds disclose herein exhibit cytotoxic effects on androgen-independent prostate cancer cells. Accordingly, such compounds are useful for treating castration-resistant prostate cancer (CRPC).

The route of administration may be selected according to a variety of factors including, but not limited to, the condition to be treated, the formulation and/or device used, the patient to be treated, and the like. Routes of administration useful in the disclosed methods include but are not limited to oral and parenteral routes, such as intravenous (iv), intraperitoneal (ip), rectal, topical, ophthalmic, nasal, and transdermal. Formulations for these dosage forms are described herein.

An effective amount of a subject compound may depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject (e.g., patient) being treated. For example, this may be the amount of a subject compound necessary to prevent, inhibit, reduce or relieve a disease or disorder, such as prostate cancer, in a subject. Ideally, a therapeutically effective amount of a compound is an amount sufficient to prevent, inhibit, reduce or relieve a disease or disorder that is mediated through histone demethylase JMJD2C activity in a subject without causing a substantial cytotoxic effect on other types of cells (e.g., non-cancerous cells) in the subject. For example, a therapeutically effective amount of a compound is an amount sufficient to prevent, inhibit, reduce or relieve a disease or disorder, such as prostate cancer, in a subject without causing a substantial cytotoxic effect on other types of cells (e.g., non-cancerous cells) in the subject.

Therapeutically effective doses of a subject compound or pharmaceutical composition can be determined by one of skill in the art, with a goal of achieving local (e.g., tissue) concentrations that are at least as high as the IC₅₀ of an applicable compound disclosed herein.

An example of a dosage range is from 0.1 to 200 mg/kg body weight orally in single or divided doses. In some embodiments, a dosage range is from 1.0 to 100 mg/kg body weight orally in single or divided doses, including from 1.0 to 50 mg/kg body weight, from 1.0 to 25 mg/kg body weight, from 1.0 to 10 mg/kg body weight (assuming an average body weight of approximately 70 kg; values may be adjusted accordingly for persons weighing more or less than average). For oral administration, the compositions are, for example, provided in the form of a tablet containing from about 10 to about 1000 mg of the active ingredient, such as 25 to 750 mg, or 50 to 500 mg, for example 75 mg, 100 mg, 200 mg, 250 mg, 400 mg, 500 mg, 600 mg, 750 mg, or 1000 mg of the active ingredient for the symptomatic adjustment of the dosage to the subject being treated. In certain embodiments of an oral dosage regimen, a tablet containing from 500 mg to 1000 mg active ingredient is administered once (e.g., a loading dose) followed by administration of ½ (i.e., half) dosage tablets (e.g., from 250 to 500 mg) each 6 to 24 hours for 3 days or more.

The specific dose level and frequency of dosage for any particular subject may be varied and may depend upon a variety of factors, including the activity of the subject compound, the metabolic stability and length of action of that compound, the age, body weight, general health, sex and diet of the subject, mode and time of administration, rate of excretion, drug combination, and severity of the condition of the host undergoing therapy.

Embodiments of the present disclosure also include combinations of one or more disclosed compounds with one or more other agents or therapies useful in the treatment of a disease or disorder. In certain instances, the disease or disorder is cell proliferative disorder, such as prostate cancer. For example, one or more disclosed compounds may be administered in combination with therapeutically effective doses of other medicinal and pharmaceutical agents, or in combination other non-medicinal therapies, such as hormone or radiation therapy. The term “administration in combination with” refers to both concurrent and sequential administration of the active agents.

In some embodiments, a suitable subject for treatment with one or more of the compounds described herein is a subject, e.g., a mammalian subject, e.g. a human subject, suffering from castration-resistant prostate cancer or at risk of developing castration-resistant prostate cancer. For example, a suitable subject may be one who has been diagnosed as having castration-resistant prostate cancer or at risk of developing castration-resistant prostate cancer. In some embodiments, a suitable subject may be one who has undergone androgen deprivation treatment but exhibits progressive signs of disease following such treatment.

Aspects, including embodiments, of the present subject matter described above may be beneficial alone or in combination, with one or more other aspects or embodiments. This is intended to provide support for all such combinations of aspects.

### EXAMPLES

The following example is put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used.

### Example 1

### Synthesis of Compound SD70-17

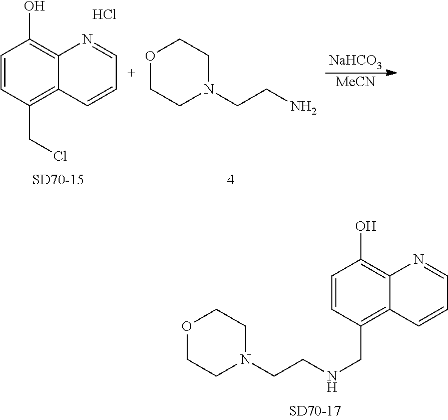

SD70-15 (0.40 g, 1.7 mmol) and a 2-morpholinoethylamine (4, 0.27 ml, 2.1 mmol) were dissolved in acetonitrile, to which NaHCO₃ (0.17 g, 2.1 mmol) was added. The mixtures were stirred at room temperature for 10 h and then concentrated in vacuo. Chromatography on silica gel afforded SD70-17.

### Example 2

### Synthesis of Compound SD70-18

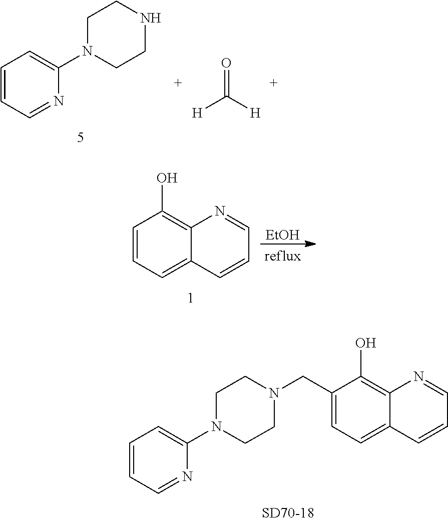 [SMILES: "EtOH" ; c1ccc(N2CCNCC2)nc1 ; C=O ; Oc1cccc2cccnc12 ; Oc1c(CN2CCN(c3ccccn3)CC2)ccc2cccnc12]

8-Hydroxyquinoline (1, 2.0 g, 14 mmol), paraformaldehyde (0.93 g, 31 mmol), and 4-(2-pyridinyl)piperazine (5, 2.4 mL, 16 mmol) were dissolved in ethanol (125 mL) and then refluxed for 4 hours. The reaction mixtures were cooled to room temperature, concentrated under reduced pressure, and chromatographed on silica gel to afford SD70-18.

### Example 3

### Synthesis of Compound SD70-19

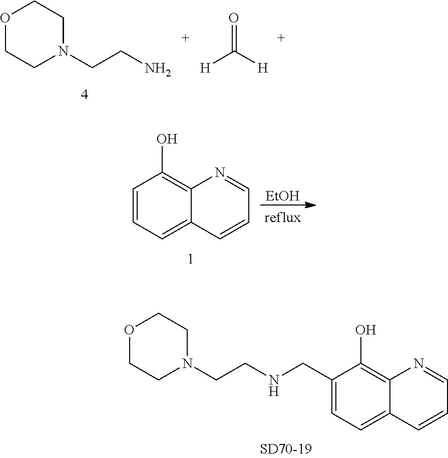 [SMILES: "EtOH" ; NCCN1CCOCC1 ; C=O ; Oc1cccc2cccnc12 ; Oc1c(CNCCN2CCOCC2)ccc2cccnc12]

8-Hydroxyquinoline (1, 2.0 g, 14 mmol), paraformaldehyde (0.93 g, 31 mmol), and 2-morpholinoethylamine (4, 2.7 ml, 21 mmol) were dissolved in ethanol (125 mL) and then refluxed for 4 hours. The reaction mixtures were cooled to room temperature, concentrated under reduced pressure, and chromatographed on silica gel to afford SD70-19.

### Example 4

### Synthesis of Compound SD70-21

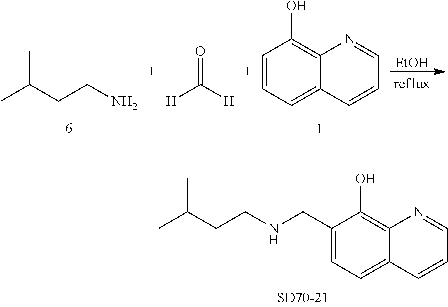

8-Hydroxyquinoline (1, 2.0 g, 14 mmol), paraformaldehyde (0.93 g, 31 mmol), and isoamylamine (6, 1.8 ml, 15 mmol) were dissolved in ethanol (125 mL) and then refluxed for 4 hours. The reaction mixtures were cooled to room temperature, concentrated under reduced pressure, and chromatographed on silica gel to afford SD70-21.

### Example 5

### Effect of Selected 8-hydroquinoline Derivatives on Androgen-Independent Prostate Cancer Cells

Cell viability assays were performed on three representative prostate cancer cell lines (i.e., CWR22Rvl, PC3 and DU145) to determine the effect of treatment with a series of 8-hydroxyquinoline derivatives.

### Materials and Methods

Cell viability was assessed 48 hours after compound treatment with the CellTiter-Glo® Luminescent Cell Viability Assay (Promega), according to the manufacturer's protocol. Briefly, 5000 cells were plated into 96-well cell culture plates in DMEM supplemented with 10% FBS. After overnight incubation, compounds at indicated concentrations were added and the treatment was maintained for two days. At the end of compound treatment, 5.0 μL of CellTiter-Glo® Reagent was added to each well. Plates were returned to the incubator for 2 hours. Plates were then removed from the incubator and luminescence was measured in a standard luminescence plate reader. The luminescence signal was then used to calculate % of survival normalized to DMSO control treatment.

### Results

The results of the cell viability assays are provided below in Table 1.

TABLE 1
Cytotoxic effects of 8-hydroxyquinoline derivatives
on androgen-independent prostate cancer cells

| | | % CWR22Rv1 | % PC3 | % DU145 |
|---|---|---|---|---|
| | | Survival | Survival | Survival |
| | | (Compound | (Compound | (Compound |
| Compound | Compound Identification and Source | at 10 μM) | at 2 μM) | at 2 μM) |
| DMSO | | 100 | 100 | 100 |
| SD70 | N-(furan-2-yl(8-hydroxyquinolin-7- | 9 | 14 | 26 |
| | yl)methyl)isobutyramide (available | | | |
| | from Chem Div, Inc., Catalog No. | | | |
| | K839-0073) | | | |
| SD70-1 | N-((5-chloro-8-hydroxyquinolin-7- | 93 | 47 | 87 |
| | yl)(furan-2-yl)methyl)propionamide | | | |
| | (available from ChemBridge Corp., | | | |
| | Catalog No. 6955265) | | | |
| SD70-2 | N-((5-chloro-8-hydroxyquinolin-7- | 100 | 9 | 32 |
| | yl)(furan-2-yl)methyl)isobutyramide | | | |
| | (available from ChemBridge Corp., | | | |
| | Catalog No. 7119963) | | | |
| SD70-3 | N-((5-chloro-8-hydroxyquinolin-7- | 110 | 88 | 110 |
| | yl)(furan-2-yl)methyl)acetamide | | | |
| | (available from ChemBridge Corp., | | | |
| | Catalog No. 6968435) | | | |
| SD70-4 | N-((5-chloro-8-hydroxyquinolin-7- | 110 | 7 | 29 |
| | yl)(furan-2-yl)methyl)butyramide | | | |
| | (available from ChemBridge Corp., | | | |
| | Catalog No. 7203222) | | | |
| SD70-5 | N-((5-chloro-8-hydroxyquinolin-7- | 94 | 23 | 31 |
| | yl)(furan-2-yl)methyl)nicotinamide | | | |
| | (available from ChemBridge Corp., | | | |
| | Catalog No. 6403782) | | | |
| SD70-6 | N-((5-chloro-8-hydroxyquinolin-7- | 21 | 24 | 50 |
| | yl)(furan-2-yl)methyl)-4- | | | |
| | methoxybenzamide (available from | | | |
| | ChemBridge Corp., Catalog No. | | | |
| | 6383468) | | | |
| SD70-7 | N-((5-chloro-8-hydroxyquinolin-7- | 28 | 21 | 21 |
| | yl)(furan-2-yl)methyl)-2- | | | |
| | phenoxyacetamide (available from | | | |
| | ChemBridge Corp., Catalog No. | | | |
| | 6420252) | | | |
| SD70-8 | N-((5-chloro-8-hydroxyquinolin-7- | 10 | 61 | 42 |
| | yl)(2- | | | |
| | methoxyphenyl)methyl)propionamide | | | |
| | (available from ChemBridge Corp., | | | |
| | Catalog No. 6983050) | | | |
| SD70-9 | N-((5-chloro-8-hydroxyquinolin-7- | 18 | 21 | 10 |
| | yl)(3,4- | | | |
| | dimethoxyphenyl)methyl)propionamide | | | |
| | (available from ChemBridge Corp., | | | |
| | Catalog No. 6988304) | | | |
| SD70-10 | N-(furan-2-yl(8-hydroxy-5- | 110 | 93 | 100 |
| | nitroquinolin-7-yl)methyl)propionamide | | | |
| | (available from ChemBridge Corp., | | | |
| | Catalog No. 6993246) | | | |
| SD70-11 | N-(furan-2-yl(8-hydroxyquinolin-7- | 16 | 79 | 120 |
| | yl)methyl)propionamide (available from | | | |
| | ChemBridge Corp., Catalog No. | | | |
| | 5927497) | | | |
| SD70-15 | 5-(chloromethyl)quinolin-8-ol | 91 | 96 | 100 |
| | hydrochloride (available from Matrix | | | |
| | Scientific, Inc., Catalog No. 060183) | | | |
| SD70-16 | 5-(benzyloxymethyl)quinolin-8-ol | 20 | 14 | 19 |
| | (available from Chem Div, Inc., Catalog | | | |
| | No. BB57-4916) | | | |
| SD70-17 | 5-((2- | 7 | 98 | 100 |
| | morpholinoethylamino)methyl)quinolin- | | | |
| | 8-ol | | | |
| SD70-18 | 7-((4-(pyridin-2-yl)piperazin-1- | 27 | 125 | 110 |
| | yl)methyl)quinolin-8-ol | | | |
| SD70-19 | 7-((2- | 107 | 98 | 94 |
| | morpholinoethylamino)methyl)quinolin- | | | |
| | 8-ol | | | |
| SD70-20 | 7-(piperidin-1-ylmethyl)quinolin-8-ol | 99 | 98 | 88 |
| | (available from Sigma-Aldrich, Catalog | | | |
| | No. CDS017373) | | | |
| SD70-21 | 7-((isopentylamino)methyl)quinolin-8- | 93 | 93 | 100 |
| | ol | | | |
| SD70-22 | N-((8-hydroxyquinolin-7-yl)(4- | 18 | 13 | 21 |
| | methoxyphenyl)methyl)isobutyramide | | | |
| | isobutyramide (available from Chem | | | |
| | Div, Inc., Catalog No. K839-0080) | | | |
| SD70-23 | N-(furan-2-yl(8-methoxyquinolin-7- | 96 | N/A | N/A |
| | yl)methyl)isobutyramide | | | |
| SD70-24 | N-((3,4-dimethoxyphenyl)(8- | 12 | N/A | N/A |
| | hydroxyquinolin-7- | | | |
| | yl)methyl)isobutyramide (available | | | |
| | from ChemBridge Corp., Catalog No. | | | |
| | 5930428) | | | |
| SD70-25 | N-((8-hydroxyquinolin-7-yl)(4- | 16 | N/A | N/A |
| | nitrophenyl)methyl)isobutyramide | | | |
| | (available from Ambinter Stock | | | |
| | Screening Collection (Greenpharma | | | |
| | S.A.S.), Catalog No. Amb10820457) | | | |

What is claimed is:

1. A compound of Formula I: 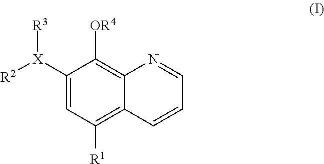
wherein
X is CH or a bond;
R¹ is selected from H, alkyl and substituted alkyl;
R² is selected from H, alkyl, substituted alkyl, amino, substituted amino, cycloalkyl, substituted cycloalkyl, heterocyclyl and substituted heterocyclyl;
R³ is selected from H, alkyl, substituted alkyl, cycloalkyl, substituted cycloalkyl, heterocyclyl, substituted heterocyclyl, aryl, substituted aryl, heteroaryl and substituted heteroaryl; and
R⁴ is selected from H, alkyl and substituted alkyl,
or a salt or stereoisomer thereof,
wherein
if X is a bond, then R² is H, R³ is absent and R¹ is aminoalkyl or substituted aminoalkyl, and
if R³ is heteroaryl or substituted aryl, then R⁴ is alkyl or substituted alkyl.

2. The compound of claim 1, wherein X is CH and R¹ is H.

3. The compound of claim 1, wherein X is a bond, R² is H, R³ is absent and R¹ is aminoalkyl or substituted aminoalkyl.

4. The compound of claim 1, wherein R¹ and R³ are each H and R² is selected from amino, substituted amino, cycloalkyl, substituted cycloalkyl, heterocyclyl and substituted heterocyclyl.

5. The compound of claim 1, wherein R¹ is H, R² amino or substituted amino, R³ is selected from aryl, substituted aryl, heteroaryl and substituted heteroaryl, and R⁴ is alkyl or substituted alkyl.

6. The compound of claim 1, wherein R⁴ is H.

7. The compound of claim 1, wherein R⁴ is alkyl or substituted alkyl.

8. The compound of claim 1, wherein the compound is a compound of Formula Ia:

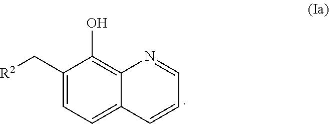

9. The compound of claim 8, wherein R² is selected from amino, substituted amino, heterocyclyl and substituted heterocyclyl.

10. The compound of claim 1, wherein the compound is a compound of Formula Ib:

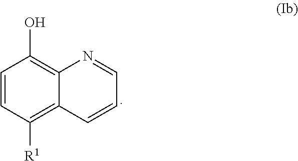

11. The compound of claim 10, wherein R¹ is aminoalkyl or substituted aminoalkyl.

12. The compound of claim 1, wherein the compound is a compound of Formula Ic:

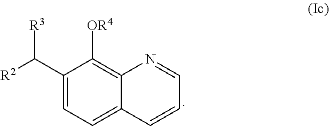

13. The compound of claim 12, wherein R² is amino or substituted amino, R³ is selected from aryl, substituted aryl, heteroaryl and substituted heteroaryl, and R⁴ is alkyl or substituted alkyl.

14. The compound of claim 1, wherein the compound is selected from:
5-((2-morpholinoethylamino)methyl)quinolin-8-ol (Compound SD70-17);
7-((4-(pyridin-2-yl)piperazin-1-yl)methyl)quinolin-8-ol (Compound SD70-18);
7-((2-morpholinoethylamino)methyl)quinolin-8-ol (Compound SD70-19); and
7-((isopentylamino)methyl)quinolin-8-ol (Compound SD70-21).

15. A pharmaceutical composition comprising: a compound selected from:
N-(furan-2-yl(8-hydroxyquinolin-7-yl)methyl)isobutyramide (Compound SD70);
N-((5-chloro-8-hydroxyquinolin-7-yl)(furan-2-yl)methyl)propionamide (Compound SD70-1);
N-((5-chloro-8-hydroxyquinolin-7-yl)(furan-2-yl)methyl)isobutyramide (Compound SD70-2);
N-((5-chloro-8-hydroxyquinolin-7-yl)(furan-2-yl)methyl)acetamide (Compound SD70-3);
N-((5-chloro-8-hydroxyquinolin-7-yl)(furan-2-yl)methyl)butyramide (Compound SD70-4);
N-((5-chloro-8-hydroxyquinolin-7-yl)(furan-2-yl)methyl)nicotinamide (Compound SD70-5);
N-((5-chloro-8-hydroxyquinolin-7-yl)(furan-2-yl)methyl)-4-methoxybenzamide (Compound SD70-6);
N-((5-chloro-8-hydroxyquinolin-7-yl)(furan-2-yl)methyl)-2-phenoxyacetamide (Compound SD70-7);
N-((5-chloro-8-hydroxyquinolin-7-yl)(2-methoxyphenyl)methyl)propionamide (Compound SD70-8);
N-((5-chloro-8-hydroxyquinolin-7-yl)(3,4-dimethoxyphenyl)methyl)propionamide (Compound SD70-9);
N-(furan-2-yl(8-hydroxy-5-nitroquinolin-7-yl)methyl)propionamide (Compound SD70-10);
N-(furan-2-yl(8-hydroxyquinolin-7-yl)methyl)propionamide (Compound SD70-11);
5-(chloromethyl)quinolin-8-ol hydrochloride (Compound SD70-15);
5-((2-morpholinoethylamino)methyl)quinolin-8-ol (Compound SD70-17);
7-((4-(pyridin-2-yl)piperazin-1-yl)methyl)quinolin-8-ol (Compound SD70-18);
7-((2-morpholinoethylamino)methyl)quinolin-8-ol (Compound SD70-19);
7-(piperidin-1-ylmethyl)quinolin-8-ol (Compound SD70-20);
7-((isopentylamino)methyl)quinolin-8-ol (Compound SD70-21);
N-((8-hydroxyquinolin-7-yl)(4-methoxyphenyl)methyl)isobutyramide (Compound SD70-22);
N-((3,4-dimethoxyphenyl)(8-hydroxyquinolin-7-yl)methyl)isobutyramide (Compound SD70-24); and
N-((8-hydroxyquinolin-7-yl)(4-nitrophenyl)methyl)isobutyramide (Compound SD70-25); and
a pharmaceutically acceptable excipient.

16. A method of inhibiting histone demethylase JMJD2C, the method comprising: administering to a subject an effective amount of a compound of any one of claims 1 to 14.

17. A method of inhibiting histone demethylase JMJD2C, the method comprising: administering to a subject an effective amount of a pharmaceutical composition of claim 15.

18. A method of treating prostate cancer, the method comprising:
administering to a subject an effective amount of a compound of any one of claims 1 to 14.

19. A method of treating prostate cancer, the method comprising:
administering to a subject an effective amount of a pharmaceutical composition of claim 15.
